(19) United States
(12) Patent Application Publication
(10) Pub. No.: US 2017/0035683 A1
(43) Pub. Date: Feb. 9, 2017

(54) NON-AQUEOUS HAIR BLEACHING AND COLORING COMPOSITIONS, METHODS OF MANUFACTURING SAME AND METHODS OF USE

(71) Applicant: Bright International Corp., Coolidge, AZ (US)

(72) Inventors: Walter B. Shepherd, Jr., Gilbert, AZ (US); Maen Anthony Bibars, Queen Creek, AZ (US)

(73) Assignee: Bright International Corp., Coolidge, AZ (US)

(21) Appl. No.: 15/304,466

(22) PCT Filed: Apr. 14, 2015
(86) PCT No.: PCT/US2015/025809

§ 371 Date: Oct. 14, 2016

Related U.S. Application Data
(60) Provisional application No. 61/979,335, filed on Apr. 14, 2014.

(51) Int. Cl. A61K 8/92; A61Q 5/06; A61K 8/73; A61K 8/23; A61K 8/02; A61Q 5/08; A61K 8/25

(52) U.S. Cl. CPC A61K 8/92; A61Q 5/08; A61Q 5/065; A61K 8/25; A61K 8/23; A61K 8/0241; A61K 8/73; A61K 2800/30; A61K 2800/48; A61K 2800/882; A61K 2800/31; A61K 2800/43

(57) ABSTRACT

The present disclosure describes non-aqueous hair bleaching and coloring compositions, method of manufacturing same, and methods of use. In various embodiments, non-aqueous hair bleaching and coloring compositions comprise silicate, silica, peroxy salt, and a dye dispersed in a hydrocarbon carrier. Compositions according to the present disclosure comprise ingredients having strictly controlled particle sizes of less than about 0.180 mm average size (passing through Tyler 80 mesh screen size), alkalinities less than about 10% Na₂O titratable to pH ˜4.5, and viscosities of at least about 1,500,000 cps at 25° C.

### CROSS REFERENCE TO RELATED APPLICATIONS

This application is a U.S. National Stage patent application of PCT Patent Application PCT/US15/25809, filed on Apr. 14, 2015, entitled “NON-AQUEOUS HAIR BLEACHING AND COLORING COMPOSITIONS, METHODS OF MANUFACTURING SAME AND METHODS OF USE,” which claims priority to U.S. Provisional Patent Application Ser. No. 61/979,335 filed on Apr. 14, 2014, which is incorporated herein by reference in its entirety.

### FIELD OF THE INVENTION

The present disclosure relates generally to hair treatment compositions and in particular to non-aqueous compositions for the simultaneous bleaching and coloring of hair.

### BACKGROUND OF THE INVENTION

Keratin fibers may be simultaneously bleached and colored in a single operation to simplify and shorten the length of time required for hair beautification. Although compositions capable of simultaneous bleaching and coloring require activation just prior to use, such as by mixing of a bleaching and coloring formulation with an aqueous hydrogen peroxide formulation, it is nonetheless convenient to have the bleaching and coloring agents in a single composition prior to activation.

Typically, a beaching and coloring formulation, prior to mixing with aqueous hydrogen peroxide, is in the form of a dry powder. This dry physical form avoids, or at least slows down, decomposition of the dyes, which occurs through reaction with the peroxy salts present in the bleaching and coloring composition. Such storage instability is greatly augmented when a bleaching and coloring composition is in liquid form. In the dry state, the peroxy salts, such as persulfates, are incapable of reacting with the dyes present while the composition remains inactivated and in storage. However, dry bleaching and coloring compositions are dusty, and are potential eye and inhalation hazards. Further, mixing a powdered bleaching and coloring composition with aqueous hydrogen peroxide prior to application to the hair often creates an inhomogeneous 2-phase (powder/liquid) suspension that easily separates during the time required to apply to the hair. A powdery bleaching and coloring composition can also be difficult to wet with the aqueous hydrogen peroxide solution, and usually clumps during mixing. In general, there is a need for bleaching and coloring compositions in physical forms other than dusty inconvenient powders.

A separate problem is that in Europe, Commission Directive 2007/54/EC dated Aug. 29, 2007 banned 85 hair dyes from use in Europe, many of these being basic dyes and direct dyes. As updated in December of 2013, only 97 substances remained allowed for restricted use in hair dye products in the EU. Thus, bleaching and coloring compositions targeted for market in the EU need to be limited to these allowed dyes.

Although hair bleaches are available in paste form, and powdered hair bleaching and coloring compositions are known, the need for hair bleaching and coloring compositions in paste-like form, and which incorporate only those hair dyes acceptable for use in the EU, remains unfulfilled.

Therefore, new, hair bleaching and coloring compositions in paste-form, which incorporate only hair dyes acceptable for use in the EU, are needed.

### SUMMARY OF THE INVENTION

Various embodiments of the present disclosure generally comprise non-aqueous hair bleaching and coloring compositions in paste form, methods of manufacturing same, and methods of use.

Various embodiments of the present disclosure comprise non-aqueous hair bleaching and coloring composition comprising (a) a silicate; (b) a silica; (c) a peroxy salt; (d) a hydrocarbon; and (e) a dye, wherein the total alkalinity of the non-aqueous bleach dye composition is less than about 10% Na₂O titratable to pH ˜4.5, and wherein the viscosity of the non-aqueous bleach dye composition is at least about 1,500,000 cps. In various embodiments of the present disclosure, non-aqueous hair bleaching and coloring compositions may further comprise any combination of a polymer thickener, a microbially-derived polysaccharide, a surfactant, a chelant, and various adjuvants such as, for example, fragrances, stabilizers, preservatives, uv inhibitors and/or conditioners.

In various embodiments, hair bleaching and coloring compositions comprise a dye selected from the group consisting of acid (anionic) dyes, nonionic dyes, cationic dyes, and mixtures thereof. In various embodiments, hair bleaching and coloring compositions comprise an acid dye. In various embodiments, compositions according to the present disclosure comprise nonionic dyes. In various embodiments, hair bleaching and coloring composition comprise at least one acid dye and at least one nonionic dye. For example, in various embodiments of the present disclosure, non-aqueous hair bleaching and coloring compositions comprise a dye selected from the group consisting of Acid Yellow 1, Acid Yellow 24, Acid Red 92, Acid Violet 43, Disperse Red 17, tetrabromophenol blue, and mixtures thereof.

Various embodiments of the present disclosure comprise methods of manufacturing non-aqueous hair bleaching and coloring composition, with the method comprising: (1) producing a powder premix by blending, at ambient temperature, a silicate, a silica, and a peroxy salt, wherein each of said silicate, silica and peroxy salt has an average particle size of less than about 0.18 mm or comprises only particles that pass through an 80 mesh Tyler Screen; (2) producing a liquid premix by mixing and heating at from about 95° to about 125° C. a hydrocarbon, a dye and optionally a surfactant; (3) producing a congealed liquid premix by cooling said liquid premix to about ambient temperature to about 65° C. until said liquid premix congeals; and (4) combining said powder premix with said congealed liquid premix at from about ambient temperature to about 50° C. while mixing to obtain said non-aqueous hair bleaching and coloring composition in the form of a paste. In various embodiments, the dye may be combined into the powder premix and/or the liquid premix.

Various embodiments of the present disclosure comprise methods for simultaneous bleaching and coloring of hair, with the method comprising mixing a non-aqueous bleaching and coloring composition with an aqueous peroxide solution to create an activated mixture, and applying the activated mixture to hair.

### DETAILED DESCRIPTION OF THE INVENTION

The following description is of various exemplary embodiments only, and is not intended to limit the scope, applicability or configuration of the present disclosure in any way. Rather, the following description is intended to provide a convenient illustration for implementing various embodiments including the best mode. As will become apparent, various changes may be made in the function and arrangement of the elements described in these embodiments without departing from principles of the present disclosure.

As described in more detail herein, various embodiments of the present disclosure generally comprise non-aqueous hair bleaching and coloring compositions, methods of manufacturing same, and methods of use.

As described in more detail herein, various embodiments of the present disclosure generally comprise a non-aqueous hair bleaching and coloring composition comprising (a) a silicate; (b) a silica; (c) a peroxy salt; (d) a hydrocarbon; and (e) a dye, wherein the total alkalinity of the non-aqueous bleach dye composition is less than about 10% Na₂O titratable to pH=4.5, and wherein the viscosity of the non-aqueous bleach dye composition is at least about 1,500,000 cps. In various embodiments of the present disclosure, non-aqueous hair bleaching and coloring compositions may further comprise any combination of polymer thickener, microbially-derived polysaccharide, surfactant, chelant, and various adjuvants such as, for example, fragrances, stabilizers, preservatives, uv inhibitors and/or conditioners. In various embodiments, the hydrocarbon can comprise any combination of liquid and waxy hydrocarbons.

As described in more detail herein, various embodiments of the present disclosure generally comprise a method of manufacturing a non-aqueous hair bleaching and coloring composition comprising: producing a powder premix comprising a mixture of silicate, silica and peroxy salt; producing a liquid premix comprising a mixture of hydrocarbon and dye; and combining the powder premix and the liquid premix to produce the non-aqueous hair bleaching and coloring composition having a paste-like consistency.

In various embodiments, non-aqueous hair bleaching and coloring compositions in accordance with the present disclosure are paste-like, resembling toothpaste or other similar compositions wherein finely powdered ingredients are stably dispersed and suspended in carriers such as polymer-thickened gels or hydrocarbons. In various embodiments, non-aqueous hair bleaching and coloring compositions in accordance with the present disclosure are pastes with high viscosity, such as for example, from about 1,500,000 to about 6,500,000 centipoise (cps).

Definitions and General Parameters

As used herein, the term “silicate” broadly refers to any silicon containing inorganic species, such as for example, an alkali metal and/or alkaline earth silicate, an alkali metal and/or alkaline earth metasilicate, an alkali metal and/or alkaline earth disilicate, and any combinations thereof. Non-aqueous hair bleaching and coloring compositions in accordance to the present disclosure comprise any one or combination of alkali metal and/or alkaline earth silicates, alkali metal and/or alkaline earth metasilicates, and/or alkali metal and/or alkaline earth di silicates.

As used herein, the term “particle size” is the physical size characteristics of the various dry/powdered ingredients incorporated in the non-aqueous hair bleaching and coloring compositions in accordance to the present disclosure. A particle size for a particular dry ingredient used herein may be stated as an average particle diameter (e.g., in units of mm, μm or nm), and/or as a particular mesh or sieve size. For example, particle size is commonly expressed in terms of either U.S. Sieve Size or Tyler Screen Mesh. Sieving or screening is a method of separating a mixture of particles into two or more size fractions. By using a single screen, oversized particles are trapped above the chosen screen while undersized particles pass through the screen. By stacking screens, particles can be separated into one of more fractions, each separable fraction remaining trapped between any two screens. In cases where certain raw materials for use herein are obtained commercially in granulate form, or in any other undesirable size and/or form, the material may be milled or ground by any means necessary prior to sieving or screening.

In various embodiments, non-aqueous hair bleaching and coloring compositions according to the present disclosure comprise various powdered ingredients having average particle size of less than about 0.20 mm. In various embodiments, non-aqueous hair bleaching and coloring compositions according to the present disclosure comprise various powdered ingredients having average particle size of less than about 0.18 mm. In various embodiments, non-aqueous hair bleaching and coloring compositions according to the present disclosure comprise various powdered ingredients that pass through an 80 mesh Tyler Screen Mesh. An 80 mesh size Tyler Mesh corresponds to a No. 80 U.S. Sieve Size, which has openings of 0.177 mm (or 0.0070 inches). In various embodiments, non-aqueous hair bleaching and coloring compositions according to the present disclosure comprises silicate, silica and peroxy salts, each ingredient having an average particle size of less than about 0.18 mm, or comprising only those particles capable of passing through an 80 mesh Tyler Screen Mesh.

As used herein, the term “non-aqueous” describes non-aqueous hair bleaching and coloring compositions in accordance to the present disclosure that are mostly free of water, but not necessarily completely anhydrous. Small amounts of water may be purposely added to the non-aqueous compositions of the present disclosure in certain embodiments, and various ingredients may contain some water, such as water of hydration, meaning their use will necessarily cause the final non-aqueous hair bleaching and coloring composition to contain at least some water. As an example, certain commercially available silicates contain 19% by weight water even though provided in powdered (and apparently dry) form. Using 25% by weight of such a silicate, for example, would result in about 4.75% by weight water incorporated into the non-aqueous hair bleaching and coloring composition.

In various embodiments, non-aqueous hair bleaching and coloring compositions in accordance to the present disclosure may comprise less than about 10% by weight water, based on the total weight of the composition. In various embodiments, non-aqueous hair bleaching and coloring compositions in accordance to the present disclosure may comprise less than about 7% by weight water, based on the total weight of the composition. In various embodiments, non-aqueous hair bleaching and coloring compositions in accordance to the present disclosure may comprise less than about 5% by weight water, based on the total weight of the composition. In various embodiments, non-aqueous hair bleaching and coloring compositions in accordance to the present disclosure may comprise less than about 2% by weight water, based on the total weight of the composition. In various embodiments, non-aqueous hair bleaching and coloring compositions in accordance to the present disclosure may be essentially anhydrous. In various embodiments, non-aqueous hair bleaching and coloring compositions in accordance to the present disclosure may be completely anhydrous.

As used herein, the term “percent (%) alkalinity,” or “% Na₂O-titratable alkalinity” refers to the capacity for non-aqueous hair bleaching and coloring compositions in accordance to the present disclosure to neutralize an acid when titrated, (i.e. titratable alkalinity). Percent alkalinity herein reports % Na₂O, as determined by titrating a non-aqueous hair bleaching and coloring compositions of the present disclosure to an endpoint (EP) of pH ˜4.5 with a dilute acid, such as for example 1.0N or 0.1N hydrochloric acid (HCl) or sulfuric acid (H₂SO₄). The endpoint is understood to be “approximately 4.5” (or “˜4.5”) because, as known by those skilled in the art, these titrations are normally conducted using an automatic titrator (called an “autotitrator”), and the software of the instrument determines, as the greatest change in the slope of the titration curve, where the endpoint is on the titration curve.

“% Na₂O-titratable alkalinity can be calculated in accordance to the following formula:

% Alkalinity=100×(mL of HCl×Normality of HCl×3.1)/gram weight of the sample

In various embodiments, non-aqueous hair bleaching and coloring compositions in accordance to the present disclosure have less than about 10.0% Na₂O-titratable alkalinity to pH ˜4.5. In various embodiments, non-aqueous hair bleaching and coloring compositions in accordance to the present disclosure have from about 5.0% to about 10.0% Na₂O-titratable alkalinity to pH ˜4.5.

As used herein, “viscosity” refers to spindle viscosity as measured, for example, on a Brookfield Viscometer equipped with a Helipath Stand™. The Helipath Stand™ allows for the use of “T-Bar” style spindles and the measurement of very high viscosities, such as the viscosity of pastes. In various embodiments, non-aqueous hair bleaching and coloring compositions in accordance to the present disclosure are thick pastes having viscosity of at least about 1,500,000 cps when measured at 25° C. (using a T-F T-Bar spindle, @ 0.1 rpm, on a Helipath Stand™ equipped Brookfield Viscometer). In various embodiments, non-aqueous hair bleaching and coloring compositions in accordance to the present disclosure are thick pastes having viscosity of at from about 1,500,000 cps to about 6,500,000 cps when measured at 25° C. (using a T-F T-Bar spindle, @ 0.1 rpm, on a Helipath Stand™ equipped Brookfield Viscometer).

Silicate

In various embodiments, non-aqueous hair bleaching and coloring compositions in accordance to the present disclosure comprise a silicate. Silicates that find use herein include, but are not limited to, lithium, sodium, and potassium silicates, metasilicates and disilicates, and combinations thereof, including mixed lithium, sodium, and potassium salts thereof.

In various embodiments, non-aqueous hair bleaching and coloring compositions in accordance to the present disclosure may comprise an alkali metal or alkaline earth silicate (i.e., any alkali metal/alkaline earth salt of Si(OH)₄, O═Si(OH)₂, and/or (HO)₃—Si—O—Si—(OH)₃), such as, for example, lithium, sodium, magnesium, calcium and/or potassium silicate, metasilicate and/or disilicate. Alkali and alkaline earth metal silicates for use herein can be described by the general formula SiO₂:M₂O, wherein M represents an alkali or alkaline earth metal, and where a weight or molar ratio of the two oxides is specified. In various embodiments, silicates that find use in the present compositions comprise SiO₂/M₂O weight ratios of from about 0.75 to about 4. In various embodiments, silicates for use in compositions herein comprise SiO₂/M₂O weight ratios of from about 0.75 to about 2.25.

Commercially available silicates that find use in the non-aqueous hair bleaching and coloring compositions according to the present disclosure include, but are not limited to, a variety of powdered silicates such as: Britesil® C20 Hydrous Sodium Silicate available from PQ Corporation, which is a powdered sodium silicate having a SiO₂/M₂O weight ratio of about 2.0; Britesil® C24 Hydrous Sodium Silicate available from PQ Corporation, which is a powdered sodium silicate having a SiO₂/M₂O weight ratio of about 2.4; Britesil® H 265 LC Hydrous Sodium Silicate available from PQ Corporation, which is a powdered sodium silicate having a SiO₂/M₂O weight ratio of about 2.65; Britesil® H20 Hydrous Sodium Silicate available from PQ Corporation, which is a powdered sodium silicate having a SiO₂/M₂O weight ratio of about 2.0; Britesil® H24 Hydrous Sodium Silicate available from PQ Corporation, which is a powdered sodium silicate having a SiO₂/M₂O weight ratio of about 2.4; G® Sodium Silicate available from PQ Corporation, which is a powdered sodium silicate having a SiO₂/M₂O weight ratio of about 3.22; GA™ Sodium Silicate available from PQ Corporation, which is a powdered sodium silicate having a SiO₂/M₂O weight ratio of about 2.4; GD® Sodium Silicate available from PQ Corporation, which is a powdered sodium silicate having a SiO₂/M₂O weight ratio of about 2.0; HS-240 Hydrous Sodium Silicate available from PQ Corporation, which is a powdered sodium silicate having a SiO₂/M₂O weight ratio of about 2.4; SS® Sodium Silicate available from PQ Corporation, which is a powdered sodium silicate having a SiO₂/M₂O weight ratio of about 3.22; SS® 20 Powder Sodium Silicate available from PQ Corporation, which is a powdered sodium silicate having a SiO₂/M₂O weight ratio of about 3.22; SS® 200 Powder Sodium Silicate available from PQ Corporation, which is a powdered sodium silicate having a SiO₂/M₂O weight ratio of about 3.22; SS® 22 Sodium Silicate available from PQ Corporation, which is a powdered sodium silicate having a SiO₂/M₂O weight ratio of about 3.22; SS® 65 Powder Sodium Silicate available from PQ Corporation, which is a powdered sodium silicate having a SiO₂/M₂O weight ratio of about 3.22; SS® 75 Sodium Silicate available from PQ Corporation, which is a powdered sodium silicate having a SiO₂/M₂O weight ratio of about 2.75; SS-C® 20 Powder Sodium Silicate available from PQ Corporation, which is a powdered sodium silicate having a SiO₂/M₂O weight ratio of about 2.0; SS-C® 200 Powder Sodium Silicate available from PQ Corporation, which is a powdered sodium silicate having a SiO₂/M₂O weight ratio of about 2.0; SS-C® Powder Sodium Silicate available from PQ Corporation, which is a powdered sodium silicate having a SiO₂/M₂O weight ratio of about 2.0; AgSil™ 16 H Potassium Silicate available from PQ Corporation, which is a powdered potassium silicate having a SiO₂/M₂O weight ratio of about 1.6; EcoDrill™ 310 Hydrous Potassium Silicate available from PQ Corporation, which is a powdered potassium silicate having a SiO₂/M₂O weight ratio of about 1.6; EcoDrill™ 743 Hydrous Potassium Silicate available from PQ Corporation, which is a powdered potassium silicate having a SiO₂/M₂O weight ratio of about 2.5; Kasil® 2.5 Flake Potassium Silicate available from PQ Corporation, which is a flaked potassium silicate having a SiO₂/M₂O weight ratio of about 2.5; Kasil® SS Powder Potassium Silicate available from PQ Corporation, which is a powdered potassium silicate having a SiO₂/M₂O weight ratio of about 2.5; and Kasolv® 16 Potassium Silicate available from PQ Corporation, which is a powdered potassium silicate having a SiO₂/M₂O weight ratio of about 1.6. These commercially available materials may be mixtures of silicate, metasilicate and/or disilicate.

Commercially available metasilicates that find use in the non-aqueous hair bleaching and coloring compositions according to the present disclosure include, but are not limited to, a variety of powdered and/or granular silicates such as: Metso Beads® 2048 Sodium Metasilicate, which is anhydrous metasilicate; and Metso Pentabead® 20 Sodium Metasilicate, pentahydrate, which is the pentahydrate of sodium metasilicate.

In various embodiments, non-aqueous hair bleaching and coloring compositions according to the present disclosure may comprise small amounts of a liquid silicate solution. For example, Kasil® 1 Potassium Silicate Solution is a 29.1% solution of potassium silicate having a SiO₂/K₂O ratio of about 2.5, which find use herein.

In various embodiments, non-aqueous hair bleaching and coloring compositions according to the present disclosure comprise silicate at from about 15% by weight to about 30% by weight total silicate, based on the total weight of the composition. In various embodiments, non-aqueous hair bleaching and coloring compositions according to the present disclosure comprise silicate at from about 15% by weight to about 25% by weight total silicate, based on the total weight of the composition. In various embodiments, non-aqueous hair bleaching and coloring compositions according to the present disclosure comprise silicate at from about 20% by weight to about 25% by weight total silicate, based on the total weight of the composition. In various embodiments, non-aqueous hair bleaching and coloring compositions according to the present disclosure comprise sodium disilicate at from about 20% by weight to about 25% by weight, based on the total weight of the composition. In various embodiments, non-aqueous hair bleaching and coloring compositions according to the present disclosure comprise a mixture of sodium silicate and sodium metasilicate at from about 20% by weight to about 25% by weight total silicate, based on the total weight of the composition.

In various embodiments, non-aqueous hair bleaching and coloring compositions according to the present disclosure comprise at least one silicate, wherein all of the silicate comprises particles having an average particle size of less than about 0.18 mm, or comprising only those particles capable of passing through an 80 mesh Tyler Screen Mesh.

Silica

In various embodiments, non-aqueous hair bleaching and coloring compositions according to the present disclosure comprise silica. Silica is typically either hydrophilic silica (e.g. untreated, fumed, hydrated or precipitated) or hydrophobic silica (from hydrophobic treatment). In various embodiments, non-aqueous hair bleaching and coloring compositions according to the present disclosure comprise at least one or combination of hydrophilic silica and/or hydrophobic silica. In various embodiments, non-aqueous hair bleaching and coloring compositions according to the present disclosure comprise silica chosen from the group consisting of hydrophilic fumed silica, hydrophobic silica, and mixtures thereof.

In various embodiments, non-aqueous hair bleaching and coloring compositions according to the present disclosure comprise hydrophilic silica. Hydrophilic silica is pure amorphous silicon dioxide, or fumed silica. Commercially available fumed silica (or silica fume) may be formed from the conversion of silicon tetrachloride with hydrogen and oxygen in the gas phase (e.g. flame process). Fumed silica traditionally finds use in liquids for rheology control, suspension, moisture resistance, viscosity stability and emulsification. Commercial fumed silica is sub-micron, (e.g. nanometer), very pure silicon dioxide (SiO₂). It may be purchased under the trade names Aerosil® and Sipernat® by Evonik-Degussa and Cab-O-Sil® from Cabot Corp. For example, commercially available hydrophilic fumed, hydrated or precipitated silica products that find use in the compositions of the present disclosure include, but are not limited to, Aerosil® 90, Aerosil® 130, Aerosil® 150, Aerosil® 200, Aerosil® 255, Aerosil® 300, Aerosil® 380, Aerosil® OX 50, Aerosil® TT 600, Aerosil® 200 F, Aerosil® 380 F, Aerosil® 200 Pharma, Aeroperl® 300/30, Aeroperl® 300 Pharma, Cab-O-Sil® M 5, Cab-O-Sil® H 5, Cab-O-Sil® HS 5, Cab-O-Sil® EH 5, Sipernat® 22, Sipernat® 22 LS, Sipernat® 50, Sipernat® 50 S, and Sipernat® 500 LS.

In various embodiments, non-aqueous hair bleaching and coloring compositions according to the present disclosure comprise from about 1% by weight to about 5% by weight hydrophilic silica, based on the total weight of the composition. In various embodiments, non-aqueous hair bleaching and coloring compositions according to the present disclosure comprise at least about 2% by weight hydrophilic silica, based on the total weight of the composition. In various embodiments, non-aqueous hair bleaching and coloring compositions according to the present disclosure comprise from about 2% by weight to about 4% by weight hydrophilic silica, based on the total weight of the composition. In various embodiments, non-aqueous hair bleaching and coloring compositions according to the present disclosure comprise from about 2% by weight to about 4% by weight Cab-O-Sil® M5 and/or Sipernat® 22 S and/or Aerosil® 200, based on the total weight of the composition. In various embodiments, non-aqueous hair bleaching and coloring compositions according to the present disclosure comprise hydrophilic fumed silica, wherein all of the silica comprises particles having an average particle size of less than about 0.18 mm, or comprising only those particles capable of passing through an 80 mesh Tyler Screen Mesh.

In various embodiments, non-aqueous hair bleaching and coloring compositions according to the present disclosure comprise hydrophobic silica. Hydrophobic silica can be made by reaction of the Si—OH groups in the silica with an alkylchlorosilane, polydimethylsiloxane, or other reagent, or by plasma polymer deposition, to derivatize (e.g. hydroxylate) the silica for overall hydrophobicity. Commercial hydrophobic silica may be purchased under the trade names Aerosil® and Sipernat® by Evonik-Degussa and Cab-O-Sil® from Cabot Corp. For example, commercially available hydrophobic silica products that find use in the compositions of the present disclosure include, but are not limited to, Aerosil® R 972; Aerosil® R 964; Aerosil® R 104; Aerosil® R 106; Aerosil® R 202; Aerosil® R 208; Aerosil® R 805; Aerosil® R 812; Aerosil® R 812 S; Aerosil® R 816; Aerosil® R 7200; Aerosil® R 8200; Aerosil® R 9200; Aerosil® R 711; Cab-O-Sil® TS-720; Cab-O-Sil® TS-710; Cab-O-Sil® TS-610; Cab-O-Sil® TS-530; Sipernat® D 10; and Sipernat® D 13.

In various embodiments, non-aqueous hair bleaching and coloring compositions according to the present disclosure comprise hydrophobic silica, wherein all of the silica comprises particles having an average particle size of less than about 0.18 mm, or comprising only those particles capable of passing through an 80 mesh Tyler Screen Mesh.

In various embodiments, non-aqueous hair bleaching and coloring compositions according to the present disclosure comprise from about 1% by weight to about 5% by weight hydrophobic silica, based on the total weight of the composition. In various embodiments, non-aqueous hair bleaching and coloring compositions according to the present disclosure comprise at least about 2% by weight hydrophobic silica, based on the total weight of the composition. In various embodiments, non-aqueous hair bleaching and coloring compositions according to the present disclosure comprise from about 2% by weight to about 4% by weight hydrophobic silica, based on the total weight of the composition. In various embodiments, non-aqueous hair bleaching and coloring compositions according to the present disclosure comprise both untreated, hydrophilic fumed silica and hydrophobic silica at from about 1% by weight to about 5% by weight total silica, based on the total weight of the composition. In various embodiments, non-aqueous hair bleaching and coloring compositions according to the present disclosure comprise hydrophobic silica, wherein all of the hydrophobic silica comprises particles having an average particle size of less than about 0.18 mm, or comprising only those particles capable of passing through an 80 mesh Tyler Screen Mesh.

Peroxy Salt

In various embodiments, non-aqueous hair bleaching and coloring compositions according to the present disclosure comprise at least one peroxy salt. Peroxy salts that find use in the present compositions include, but are not limited to, lithium, sodium, potassium, magnesium, calcium and/or ammonium persulfate, percarbonate, perborate and/or peroxide salts. Additional peroxy salts that find use in the present compositions include strontium and manganese peroxide. In various embodiments, non-aqueous hair bleaching and coloring compositions according to the present disclosure comprise at least one of ammonium persulfate, potassium persulfate and/or sodium persulfate. In various embodiments, non-aqueous hair bleaching and coloring compositions according to the present disclosure comprise at least one of ammonium percarbonate, potassium percarbonate and/or sodium percarbonate. In various embodiments, non-aqueous hair bleaching and coloring compositions according to the present disclosure comprise at least one of ammonium perborate, potassium perborate and/or sodium perborate. In various embodiments, non-aqueous hair bleaching and coloring compositions according to the present disclosure comprise at least one peroxy salt having the formula (HOO⁻)ₓM^{x+}, wherein x=1 or 2 and M is sodium, potassium and/or magnesium.

In various embodiments, non-aqueous hair bleaching and coloring compositions according to the present disclosure comprise from about 25% by weight to about 60% by weight of peroxy salt, based on the total weight of the composition. In various embodiments, non-aqueous hair bleaching and coloring compositions according to the present disclosure comprise from about 35% by weight to about 50% by weight of peroxy salt, based on the total weight of the composition. In various embodiments, non-aqueous hair bleaching and coloring compositions according to the present disclosure comprise from about 40% by weight to about 45% by weight of peroxy salt, based on the total weight of the composition. In various embodiments, non-aqueous hair bleaching and coloring compositions according to the present disclosure comprise from about 25% by weight to about 60% by weight, based on the total weight of the composition, a peroxy salt selected from the group consisting of ammonium persulfate, potassium persulfate, sodium persulfate, and mixtures thereof, wherein all of the persulfate salts comprise particles having an average particle size of less than about 0.18 mm, or comprise only those particles capable of passing through an 80 mesh Tyler Screen Mesh. In various embodiments, non-aqueous hair bleaching and coloring compositions according to the present disclosure comprise a total of from about 35% by weight to about 50% by weight, based on the total weight of the composition, at least two peroxy salts selected from the group consisting of ammonium persulfate, potassium persulfate and sodium persulfate, wherein all of the persulfate salts comprise particles having an average particle size of less than about 0.18 mm, or comprise only those particles capable of passing through an 80 mesh Tyler Screen Mesh. In various embodiments, non-aqueous hair bleaching and coloring compositions according to the present disclosure comprise a total of from about 40% by weight to about 45% by weight, based on the total weight of the composition, at least two peroxy salts selected from the group consisting of ammonium persulfate, potassium persulfate and sodium persulfate, wherein all of the persulfate salts comprise particles having an average particle size of less than about 0.18 mm, or comprise only those particles capable of passing through an 80 mesh Tyler Screen Mesh.

Hydrocarbon

In various embodiments, non-aqueous hair bleaching and coloring compositions according to the present disclosure comprise a hydrocarbon. In the present compositions, the hydrocarbon acts as the vehicle for the composition, in which various powdered ingredients are dispersed and substantially stably suspended to yield a pasty or creamy looking product of high viscosity. In various embodiments, non-aqueous hair bleaching and coloring compositions according to the present disclosure comprise C₁₀-C₁₀₀ hydrocarbon. “Hydrocarbon” as used herein is not meant to be limiting, but is meant to include mixtures of hydrocarbons. Hydrocarbon for use in the present compositions may comprise any combination of chain lengths, and may be linear or branched, saturated or partly unsaturated, cyclic, or aromatic, in any combination. Hydrocarbon for use in various embodiments of the present composition may comprise any combination of liquid and/or waxy (i.e. “waxes”) hydrocarbon materials depending on the chain lengths and degree of unsaturation and branching. Hydrocarbon for use herein may be any combination of paraffinic oils, naphthenic oils, aromatic oils, liquid alkanes and alkenes, and solid/waxy materials. Hydrocarbon for use as a carrier herein can be petroleum, animal, or vegetable sourced, and can be any “cut” or distribution of chain lengths, with any degree of branching and unsaturation. Vegetable/animal sourced hydrocarbon will typically contain only the even carbon number chains, whereas petroleum sourced hydrocarbon typically contains both the odd and even carbon number chains.

In various embodiments, non-aqueous hair bleaching and coloring compositions according to the present disclosure comprise mineral oil. Mineral oil is a petroleum-sourced paraffinic oil mixture of alkanes having a distribution of about C₁₅-C₄₀. Mineral oil for use herein may also be referred to as light mineral oil, white oil, liquid paraffin, and/or liquid petroleum.

In various embodiments, non-aqueous hair bleaching and coloring compositions according to the present disclosure comprise a hydrocarbon wax and/or jelly, that is, hydrocarbon that is not entirely liquid at ambient temperature. Such hydrocarbons include, but are not limited to, microcrystalline wax, ceresin and ozokerite waxes, beeswax, carnauba, petrolatum, mineral jelly, and petroleum jelly. Exemplary non-liquid hydrocarbons for use herein are available from Penreco/Calumet Specialty Products, amongst others. In various embodiments, non-aqueous hair bleaching and coloring compositions according to the present disclosure comprise a mixture of liquid and waxy hydrocarbons as the carrier.

In various embodiments, non-aqueous hair bleaching and coloring compositions according to the present disclosure comprise a hydrocarbon at from about 15% by weight to about 40% by weight, based on the total weight of the composition. In various embodiments, non-aqueous hair bleaching and coloring compositions according to the present disclosure comprise a hydrocarbon at from about 20% by weight to about 30% by weight, based on the total weight of the composition. In various embodiments, non-aqueous hair bleaching and coloring compositions according to the present disclosure comprise mineral oil at from about 15% by weight to about 40% by weight, based on the total weight of the composition. In various embodiments, non-aqueous hair bleaching and coloring compositions according to the present disclosure comprise mineral oil at from about 20% by weight to about 30% by weight, based on the total weight of the composition.

In various embodiments, non-aqueous hair bleaching and coloring compositions according to the present disclosure are entirely absent any organic ester compounds. Organic esters have the general formula R¹—CO₂—R², and have been used in non-aqueous bleaching and/or coloring compositions for various reasons in the past, such as solvent. For example, esters such as myristyl myristate have been used as solubilizers and for stabilizing bleaching and coloring compositions comprising dyes. It has now been discovered that the use of such organic esters in non-aqueous bleaching and coloring compositions is not needed. In various embodiments however, some esters find use in the compositions of the present invention as conditioners.

Dyes

The non-aqueous hair bleaching and coloring compositions according to the present disclosure comprise at least one dye. Hair dyes for use herein include acid dyes (also commonly known as anionic dyes), nonionic dyes (which generally include the basic dyes containing uncharged amine substituents and/or nitro groups, and the like), and cationic dyes. General structure (e.g., diazo, naphthalene, anthracine, anthraquinone, xanthene, etc.) overlaps across these categories of dyes, since it is the substituent groups (e.g. —O⁻, —CO₂⁻, —NR₂, —NO₂, —NR₃⁺, etc.) on the dye molecule that determine if the dye is anionic, nonionic or cationic, not the general structure of the dye.

In various embodiments, non-aqueous hair bleaching and coloring compositions according to the present disclosure comprise a dye chosen from the group consisting of anionic dyes, nonionic dyes, cationic dyes, and mixtures thereof.

In various embodiments, non-aqueous hair bleaching and coloring compositions according to the present disclosure comprise a dye chosen from the group consisting of anionic dyes, nonionic dyes, and mixtures thereof.

In various embodiments, non-aqueous hair bleaching and coloring compositions according to the present disclosure comprise at least one acid dye. Acid dyes are organic molecules having one or more ionizable acidic substituents, such as for example, —SO₃H, —OH, and/or —CO₂H. These dyes are also referred to as “anionic dyes” because these substituent groups can be present as anionic (negatively charged) substituents (e.g. —SO₃⁻, —O⁻, —CO₂⁻) when the dye is formulated in a sufficiently alkaline medium. Prior to the present disclosure, non-aqueous pasty compositions comprising acid dyes and peroxy salts in hydrocarbon was not described.

As mentioned above, many of the direct, basic, and mordant hair dyes have been banned in the EU, necessitating the need to develop stable and dust free hair bleaching and coloring compositions comprising acid dyes allowed for use in the EU.

Acid dyes that find use in the present compositions include, but are not limited to, the various acid dyes set out in TABLE 1 below. These and other acid dyes that find use in the compositions of the present disclosure may be sourced from American Dyestuff Corporation, Clifton, N.J., amongst other suppliers.

TABLE 1
Acid Dyes

| Name | C.I. No. | CAS No. |
|---|---|---|
| Acid Black 1 | 20470 | 1064-48-8 |
| Acid Black 172 | 15711:1 | 61847-77-6 |
| Acid Black 194 | n/a | 61931-02-0 |
| Acid Black 2 | 50420 | 1064-48-8 |
| Acid Black 234 | 30027 | 157577-99-6 |
| Acid Black 24 | 26370 | 3071-73-6 |
| Acid Blue 113 | 26360 | 3351-05-1 |
| Acid Blue 22 | 42755 | 28631-66-5 |
| Acid Blue 25 | 62055 | 6408-78-2 |
| Acid Blue 7 | 423080 | 3486-30-4 |
| Acid Blue 83 | 42660 | 6104-59-2 |
| Acid Blue 92 | 13390 | 3861-73-2 |
| Acid Brown 15 | 20190 | 5850-15-7 |
| Acid Brown 161 | n/a | 61724-13-8 |
| Acid Brown 348 | n/a | 72827-72-6 |
| Acid Brown 349 | n/a | 72827-73-7 |
| Acid Brown 440 | 34908 | 93376-15-9 |
| Acid Brown 75 | 34905 | 8011-86-7 |
| Acid Brown 97 | n/a | 61901-18-6 |
| Acid Brown 98 | 18840:2 | 12269-88-4 |
| Acid Green 20 | 20495 | 5850-39-5 |
| Acid Green 25 | 61570 | 4403-90-1 |
| Acid Orange 7 | 15510 | 633-96-5 |
| Acid Orange 12 | 15970 | 1934-20-9 |
| Acid Orange 74 | 18745 | 10127-27-2 |
| Acid Red 1 | 18050 | 3734-67-6 |
| Acid Red 4 | 14710 | n/a |
| Acid Red 14 | 14720 | 3567-69-9 |
| Acid Red 18 | 16255 | 2611-82-7 |
| Acid Red 26 | 16150 | 3761-53-3 |
| Acid Red 29 | 16570 | n/a |
| Acid Red 33 | 17208 | 3567-66-6 |
| Acid Red 44 | 16250 | n/a |
| Acid Red 51 | 45430 | n/a |
| Acid Red 52 | 45100 | 3520-42-1 |
| Acid Red 66 | 26905 | 4196-99-0 |
| Acid Red 73 | 27290 | 5413-75-2 |
| Acid Red 87 | 45380 | 17372-87-1 |
| Acid Red 88 | 15620 | 1658-56-6 |
| Acid Red 91 | 45400 | n/a |
| Acid Red 92 | 45410 | 18472-87-2 |
| Acid Red 97 | 22890 | 10169-02-5 |
| Acid Red 114 | 23635 | 6459-94-5 |
| Acid Red 388 | n/a | 37299-86-8 |
| Acid Violet 7 | 18055 | 4321-69-1 |
| Acid Violet 43 | 60730 | 4430-18-6 |
| Acid Yellow 1 | 10316 | 846-70-8 |
| Acid Yellow 7 | 56205 | n/a |
| Acid Yellow 9 | 13015 | n/a |
| Acid Yellow 17 | 18965 | 6359-89-4 |
| Acid Yellow 23 | 19140 | n/a |
| Acid Yellow 24 | 10315 | 10142-54-8 |
| Acid Yellow 36 | 13065 | 587-98-4 |
| Acid Yellow 42 | 22910 | 6375-55-9 |
| Acid Yellow 73 | 45350 | 518-47-8 |
| Tetrabromophenol Blue - Na salt | n/a | 108321-10-4 |

In various embodiments, non-aqueous hair bleaching and coloring compositions according to the present disclosure comprise at least one acid dye from TABLE 1. In various embodiments, non-aqueous hair bleaching and coloring compositions according to the present disclosure comprise at least one acid dye from TABLE 1, wherein all of the acid dyes(s) comprise particles having an average particle size of less than about 0.18 mm, or comprise only those particles capable of passing through an 80 mesh Tyler Screen Mesh.

In various embodiments, compositions in accordance with the present disclosure comprise Acid Yellow 1 dye, CAS No. 846-70-8, having the following chemical structure:

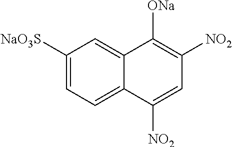

In various embodiments, compositions in accordance with the present disclosure comprise Acid Yellow 24 dye, CAS No. 10142-54-8, having the following chemical structure:

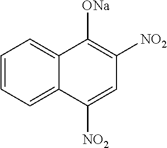

In various embodiments, compositions in accordance with the present disclosure comprise Acid Red 92 dye, CAS No. 18472-87-2, having the following chemical structure:

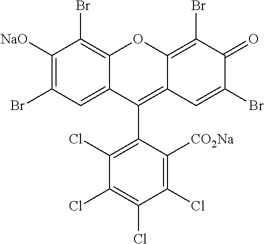

In various embodiments, compositions in accordance with the present disclosure comprise Acid Violet 43 dye, CAS No. 4430-18-6, having the following chemical structure:

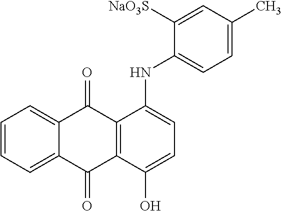

In various embodiments, compositions in accordance with the present disclosure comprise tetrabromophenol blue, sodium salt, CAS No. 108321-10-4, having the following chemical structure:

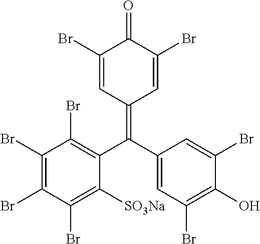

In various embodiments, compositions in accordance with the present disclosure comprise a mixture of Acid Red 92 and Acid Violet 43 dyes.

In various embodiments, compositions in accordance with the present disclosure comprise an acid dye selected from the group consisting of Acid Yellow 1, Acid Yellow 24, Acid Red 92, Acid Violet 43, tetrabromophenol blue sodium salt, and mixtures thereof.

In various embodiments, compositions in accordance with the present disclosure comprise any two of Acid Yellow 1, Acid Yellow 24, Acid Red 92, Acid Violet 43, and tetrabromophenol blue sodium salt.

In various embodiments, non-aqueous hair bleaching and coloring compositions according to the present disclosure comprise at least one nonionic dye. In various embodiments, non-aqueous hair bleaching and coloring compositions according to the present disclosure comprise at least one nonionic dye comprising particles having an average particle size of less than about 0.18 mm, or comprise only those particles capable of passing through an 80 mesh Tyler Screen Mesh.

For example, compositions in accordance with the present disclosure may comprise Disperse Red 17 (CAS No. 3179-89-3, C.I. 11210), or any other nonionic dye. Disperse Red 17 is a diazo dye having one nitro substituent and one —N(CH₂CH₂OH)₂ substituent, and is therefore a nonionic dye as discussed herein above. Nonionic dyes for use herein include, but are not limited to, many Basic and Disperse yellow, orange, red, violet, blue and black dyes, such as those available from American Dyestuff Corporation, Clifton N.J., amongst other suppliers.

In various embodiments, non-aqueous hair bleaching and coloring compositions according to the present disclosure comprise at least one dye chosen from the group consisting of acid dyes, nonionic dyes, cationic dyes, and mixtures thereof, wherein all of the dyes(s) in the composition comprise particles having an average particle size of less than about 0.18 mm, or comprise only those particles capable of passing through an 80 mesh Tyler Screen Mesh.

In various embodiments, non-aqueous hair bleaching and coloring compositions according to the present disclosure comprise at least one dye at from about 0.1% by weight to about 7% by weight, based on the total weight of the composition. In various embodiments, non-aqueous hair bleaching and coloring compositions according to the present disclosure comprise from about 0.1% by weight to about 7% by weight of a dye selected from the group consisting of anionic dyes, nonionic dyes, cationic dyes, and mixtures thereof, based on the total weight of the composition.

In various embodiments, non-aqueous hair bleaching and coloring compositions according to the present disclosure comprise at least one acid dye at from about 0.1% by weight to about 7% by weight, based on the total weight of the composition, wherein all of the acid dyes(s) comprise particles having an average particle size of less than about 0.18 mm, or comprise only those particles capable of passing through an 80 mesh Tyler Screen Mesh. In various embodiments, non-aqueous hair bleaching and coloring compositions according to the present disclosure comprise from about 0.1% by weight to about 7% by weight an acid dye selected from the group consisting of Acid Yellow 1, Acid Yellow 24, Acid Red 92, Acid Violet 43, tetrabromophenol blue-sodium salt, and mixtures thereof, based on the total weight of the composition, wherein all of the acid dyes(s) comprise particles having an average particle size of less than about 0.18 mm, or comprise only those particles capable of passing through an 80 mesh Tyler Screen Mesh.

Other dyes that find use in the non-aqueous bleaching and coloring compositions of the present disclosure are disclosed in Said, et al., U.S. Pat. Nos. 5,688,291 and 7,189,265, incorporated herein to the extent of the dye listings disclosed.

Polymer Thickener

In various embodiments, non-aqueous hair bleaching and coloring compositions according to the present disclosure may comprise at least one polymer thickener. Polymer thickeners that may find use in compositions of the present disclosure include, but are not limited to, natural, semisynthetic, or synthetic homo-polymers, random co-polymers and block co-polymers. Examples of homo-polymers include various high molecular weight polyacrylate polymers and co-polymers. Polymer thickeners can be utilized alone or in combination.

Commercially available polymer thickeners that may find use in the non-aqueous hair bleaching and coloring compositions according to the present disclosure include, but are not limited to, a variety of acrylate-based materials under the Carbopol®, Noveon® and Pemulin™ trade names from Lubrizol, such as for example, polyacrylates Carbopol® 934, 937, 940, 941, 980, 981, 2984, and 5984; acrylates/C₁₀-C₃₀ alkyl acrylate cross-polymer (Carbopol® Ultrez 10, 20, 21, and 30, Carbopol® Aqua SF-1, SF-2, and CC, Carbopol® 1342, 1382, and Pemulin™ TR-1 and TR-2); acrylates/steareth-20 itaconate co-polymer and acrylates/ceteth-20 itaconate co-polymer (e.g. from National Starch); PVM/MA decadiene cross-polymer (e.g. Stabileze® QM from Ashland, Inc.); acrylates/steareth-20 methacrylate copolymer, (e.g. Aculyn™ 22 from Dow Chemical); Acrysol™ DR-1 from Dow Chemical; acrylamide/sodium acrylate copolymer, (e.g. Hostacerin® PN 73 from Hoecsht AG); acrylic acid/acrylonitrogens copolymer (e.g. Hypan® QT-100, SR-150H, SA-100H and SR-150H from Lipo Technologies); acrylic/acrylate copolymer (Carboset® 514, 515, 525, XL-19, XL-19-X2, XI-28, XL-40, and 526 from Lubrizol); ammonium acrylates/acrylonitrogens copolymer (e.g. Hypan® SS-201 from Lipo Technologies); sodium salt of cross-linked polyacrylic acid (e.g. PNC® 430, PNC® 410, PNC® 400 from 3V-Sigma); acrylamide/sodium acrylate copolymer (e.g. Hostacerin® PN 73 from Hoechst AG); poly(acrylic acid) (the Carbopol® 400 series from Lubizol and Aquatreat® products from 3V-Sigma); dimethylaminoethyl methacrylamide and acrylamide copolymer (e.g. Salcare® SC63 from Ciba Specialties); and acrylic polymer cationic thickening agents (Synthalen® K and Synthalen® CR, and related compounds from 3V-Sigma.

Other polymer thickeners for use herein can be found in the “The Encyclopedia of Polymers and Thickeners for Cosmetics,” Cosmetics and Toiletries, Lochhead, R., pp. 95-138, Vol. 108, (May 1993).

In various embodiments, non-aqueous hair bleaching and coloring compositions according to the present disclosure may comprise at least one polymer thickener at from about 1% by weight to about 5% by weight, based on the total weight of the composition. In various embodiments, non-aqueous hair bleaching and coloring compositions according to the present disclosure may comprise at least one polymer thickener at from about 1% by weight to about 5% by weight, based on the total weight of the composition, wherein all of the polymer thickener comprises particles having an average particle size of less than about 0.18 mm, or comprises only those particles capable of passing through an 80 mesh Tyler Screen Mesh. In various embodiments, non-aqueous hair bleaching and coloring compositions according to the present disclosure may comprise at least one polyacrylate polymer thickener chosen from the group consisting of Carbopol® 934, Carbopol® 937, Carbopol® 941, Carbopol® 971, Carbopol® 980, Carbopol® 981, and mixtures thereof. In various embodiments, non-aqueous hair bleaching and coloring compositions according to the present disclosure may comprise at least one polyacrylate polymer thickener at from about 1% by weight to about 5% by weight, based on the total weight of the composition. In various embodiments, non-aqueous hair bleaching and coloring compositions according to the present disclosure may comprise at least one Carbopol® polyacrylate polymer at from about 1% by weight to about 5% by weight, based on the total weight of the composition.

Microbially-Derived Polysaccharide

In various embodiments, non-aqueous hair bleaching and coloring compositions according to the present disclosure may comprise at least one microbially-derived polysaccharide. A number of polysaccharidic materials are generated by microorganisms. Some examples include, but are not limited to, alginate, curdlane, dextran, diutan, gellan, glucan, pullulan, and xanthan. Xanthan gum is produced as an extra-cellular product in the fermentation of glucose or sucrose by the bacterium Xanthomonas campestris. Since the backbone of xanthan gum consists of two β-D-glucose units linked through the 1 and 4 positions, each monosaccharide unit in the backbone has two free hydroxyl groups (—OH groups). The side chains of the molecule consist of two mannose and one glucuronic acid monosaccharidic units. Some of these mannose units have a pyruvate ester linkage, but nonetheless, considerable numbers of free hydroxyl groups remain in the repeating side chains of the xanthan gum polymer as well as in the backbone. That being said, and not wishing to be bound by any particular theory, it remains possible that a microbially-derived polysaccharide such as xanthan gum helps emulsify and/or disperse the non-aqueous bleaching and coloring composition into the aqueous hydrogen peroxide solution prior to application to the hair, perhaps due to the presence of free hydroxyl groups.

In various embodiments, non-aqueous hair bleaching and coloring compositions according to the present disclosure may comprise a microbially-derived polysaccharide chosen from the group consisting of alginate, curdlane, dextran, diutan, gellan, glucan, pullulan, xanthan, and mixtures thereof.

In various embodiments, non-aqueous hair bleaching and coloring compositions according to the present disclosure comprise xanthan gum. Commercial xanthan gum is available from CP Kelco under the trade names Keltrol RD®, Keizan S®, Kelzan T® and Kelzan AR®. In various embodiments, non-aqueous hair bleaching and coloring compositions according to the present disclosure comprise a xanthan gum chosen from the group consisting of Keltrol RD®, Keizan S®, Kelzan T®, Kelzan AR®, and mixtures thereof.

In various embodiments, non-aqueous hair bleaching and coloring compositions according to the present disclosure may comprise at least one microbially-derived polysaccharide at from about 1% by weight to about 2% by weight, based on the total weight of the composition. In various embodiments, non-aqueous hair bleaching and coloring compositions according to the present disclosure may comprise at least one microbially-derived polysaccharide at from about 1% by weight to about 2% by weight, based on the total weight of the composition, wherein all of the microbially-derived polysaccharide comprises particles having an average particle size of less than about 0.18 mm, or comprises only those particles capable of passing through an 80 mesh Tyler Screen Mesh. In various embodiments, non-aqueous hair bleaching and coloring compositions according to the present disclosure may comprise xanthan gum at from about 1% by weight to about 2% by weight, based on the total weight of the composition. In various embodiments, non-aqueous hair bleaching and coloring compositions according to the present disclosure may comprise xanthan gum at from about 1% by weight to about 2% by weight, based on the total weight of the composition, wherein the xanthan gum comprises particles having an average particle size of less than about 0.18 mm, or comprises only those particles capable of passing through an 80 mesh Tyler Screen Mesh.

Surfactant

In various embodiments, non-aqueous hair bleaching and coloring compositions according to the present disclosure may comprise at least one surfactant. Surfactants for use in the present compositions may comprise any anionic, nonionic, cationic, or amphoteric surfactant, or combinations thereof.

Various anionic surfactants that find use in the present compositions include, but are not limited to, carboxylate salts (salts of fatty acids), alkyl benzene sulfonates, alkyl ether sulfates, alkyl sulfates, and combinations thereof. In various embodiments, non-aqueous hair bleaching and coloring compositions according to the present disclosure may comprise at least one anionic surfactant chosen from the group consisting of carboxylate salts, alkyl benzene sulfonates, alkyl ether sulfates, alkyl sulfates, and combinations thereof

Fatty acids that find use in the compositions of the present disclosure may be represented by the general formula R—COOH, wherein R represents a linear or branched alkyl or alkenyl group having between about 8 and about 26 carbons. It is understood that within the compositions herein, the free fatty acid form (i.e., the carboxylic acid) will be converted to the alkali metal salt in-situ (i.e., to the fatty acid soap, or the more formally the “carboxylate salt”), by the excess alkalinity present in the composition, due at least in part to the presence of silicate. As used herein, “soap” means salts of fatty acids. Thus, after mixing and obtaining the non-aqueous bleaching and coloring compositions of the present disclosure, the fatty acids may be present in the composition as R—COOM, wherein represents a linear or branched alkyl or alkenyl group having between about 8 and 24 carbons and M represents an alkali metal, alkaline earth metal, or ammonium ion, such as, for example, sodium, potassium, magnesium and/or NH₄⁺. Fatty acids for use herein may be derived from natural fats and oils, such as those from animal fats and greases and/or from vegetable or seed oils, for example, tallow, hydrogenated tallow, whale oil, fish oil, grease, lard, coconut oil, palm oil, palm kernel oil, olive oil, peanut oil, corn oil, sesame oil, rice bran oil, cottonseed oil, babassu oil, soybean oil, castor oil, and mixtures thereof. Fatty acids can be synthetically prepared, for example, by the oxidation of petroleum, or by hydrogenation of carbon monoxide by the Fischer-Tropsch process.

In various embodiments, fatty acids may be incorporated into the non-aqueous bleaching and coloring compositions as their carboxylate salts, thus avoiding the need for in-situ neutralization. For example, any alkali metal, alkaline earth, or ammonium salt of a C₈-C₂₆ fatty acid may be directly added to the compositions of the present disclosure. Fatty carboxylate salts that may be used herein include, but are not limited to, sodium, potassium, magnesium or ammonium caprylate (C₈), caprate (C₁₀), laurate (C₁₂), myristate (C₁₄), palmitate (C₁₆), stearate (C₁₈), arachidate (C₂₀), behenate (C₂₂), lignocerate (C₂₄), cerotate (C₂₆), and mixtures thereof, and mixed salts thereof.

In various embodiments, non-aqueous hair bleaching and coloring compositions according to the present disclosure may comprise from about 1% by weight to about 5% by weight of a C₈-C₂₆ carboxylate salt, based on the total weight of the composition. In various embodiments, non-aqueous hair bleaching and coloring compositions according to the present disclosure may comprise from about 1% by weight to about 5% by weight of a C₈-C₂₆ carboxylate salt, based on the total weight of the composition, wherein the C₈-C₂₆ carboxylate salt comprises particles having an average particle size of less than about 0.18 mm, or comprises only those particles capable of passing through an 80 mesh Tyler Screen Mesh. In various embodiments, non-aqueous hair bleaching and coloring compositions according to the present disclosure may comprise from about 1% by weight to about 5% by weight of a C₈-C₂₆ sodium carboxylate selected from the group consisting of sodium caprylate (C₈), sodium caprate (C₁₀), sodium laurate (C₁₂), sodium myristate (C₁₄), sodium palmitate (C₁₆), sodium stearate (C₁₈), sodium arachidate (C₂₀), sodium behenate (C₂₂), sodium lignocerate (C₂₄), sodium cerotate (C₂₆), and mixtures thereof, based on the total weight of the composition. In various embodiments, non-aqueous hair bleaching and coloring compositions according to the present disclosure may comprise from about 1% by weight to about 5% by weight of a C₈-C₂₆ potassium carboxylate selected from the group consisting of potassium caprylate (C₈), potassium caprate (C₁₀), potassium laurate (C₁₂), potassium myristate (C₁₄), potassium palmitate (C₁₆), potassium stearate (C₁₈), potassium arachidate (C₂₀), potassium behenate (C₂₂), potassium lignocerate (C₂₄), potassium cerotate (C₂₆), and mixtures thereof, based on the total weight of the composition. In various embodiments, non-aqueous hair bleaching and coloring compositions according to the present disclosure may comprise from about 1% by weight to about 5% by weight of a C₈-C₂₆ magnesium carboxylate selected from the group consisting of magnesium caprylate (C₈), magnesium caprate (C₁₀), magnesium laurate (C₁₂), magnesium myristate (C₁₄), magnesium palmitate (C₁₆), magnesium stearate (C₁₈), magnesium arachidate (C₂₀), magnesium behenate (C₂₂), magnesium lignocerate (C₂₄), magnesium cerotate (C₂₆), and mixtures thereof, based on the total weight of the composition.

In various embodiments, non-aqueous hair bleaching and coloring compositions according to the present disclosure may comprise a nonionic surfactant. Nonionic surfactants useful in the present compositions may include, but are not limited to, ethoxylated and/or propoxylated, primary alcohols, (i.e., alcohol alkoxylates) and amine oxides. In various embodiments, non-aqueous hair bleaching and coloring compositions according to the present disclosure may comprise a cationic surfactant. In various embodiments, non-aqueous hair bleaching and coloring compositions according to the present disclosure may comprise an amphoteric surfactant such as, for example, a betaine.

Chelant

In various embodiments, non-aqueous hair bleaching and coloring compositions according to the present disclosure may comprise a chelant. Chelants that find use in the present compositions include, but are not limited to, inorganic and organic phosphates, nitrilotriacetic acid salts (NTA), ethylenediaminetetraacetic acid salts (EDTA), ethylenediaminedisuccinic acid salts (EDDS), diethylenetriamine pentaacetic acid salts (DTPA), and combinations thereof. For example, compositions of the present disclosure may include disodium-, trisodium-, and/or tetrasodium ethylenediamine salts.

In various embodiments, non-aqueous hair bleaching and coloring compositions according to the present disclosure may comprise from about 0.5% by weight to about 5% by weight of an EDTA salt, based on the total weight of the composition. In various embodiments, non-aqueous hair bleaching and coloring compositions according to the present disclosure may comprise from about 0.5% by weight to about 5% by weight of tetrasodiumethylenetetraacetate, based on the total weight of the composition. In various embodiments, non-aqueous hair bleaching and coloring compositions according to the present disclosure may comprise from about 0.5% by weight to about 5% by weight of tetrasodiumethylenetetraacetate, based on the total weight of the composition, wherein the tetrasodiumethylenetetraacetate comprises particles having an average particle size of less than about 0.18 mm, or comprises only those particles capable of passing through an 80 mesh Tyler Screen Mesh. In various embodiments, non-aqueous hair bleaching and coloring compositions according to the present disclosure may comprise from about 0.5% by weight to about 2% by weight of tetrasodiumethylenetetraacetate, based on the total weight of the composition, wherein the tetrasodiumethylenetetraacetate comprises particles having an average particle size of less than about 0.18 mm, or comprises only those particles capable of passing through an 80 mesh Tyler Screen Mesh.

Adjuvant

In various embodiments, non-aqueous hair bleaching and coloring compositions according to the present disclosure may comprise adjuvant, such as, for example, any combination of fragrance, preservative, uv inhibitor, and/or conditioner. In various embodiments, non-aqueous hair bleaching and coloring compositions according to the present disclosure may comprise adjuvant totaling from about 0.1% by weight to about 2.0% by weight, based on the total weight of the composition. In various embodiments, non-aqueous hair bleaching and coloring compositions according to the present disclosure may comprise adjuvant, wherein any powdered adjuvant comprises particles having an average particle size of less than about 0.18 mm, or comprises only those particles capable of passing through an 80 mesh Tyler Screen Mesh. In various embodiments, non-aqueous hair bleaching and coloring compositions according to the present disclosure may comprise liquid adjuvant chosen from the group consisting of fragrance, preservative, uv inhibitor, conditioner, and mixtures thereof. In various embodiments, non-aqueous hair bleaching and coloring compositions according to the present disclosure may comprise powdered adjuvant chosen from the group consisting of fragrance, preservative, uv inhibitor, conditioner, and mixtures thereof. In various embodiments, non-aqueous hair bleaching and coloring compositions according to the present disclosure may comprise any combination of various liquid and/or powdered adjuvant chosen from the group consisting of fragrance, preservative, uv inhibitor, conditioner, and mixtures thereof.

Various conditioners for use in the compositions of the present disclosure include, but are not limited to, the Celquat® cationic polymers, the Celquat® polyquaternium-4-polymers, and the Arquad® PC mono-, di-, and tri-alkyl quaternary conditioning agents, all available from AkzoNobel; waxes and microcrystalline waxes from various suppliers; and various silicone conditioners available from Dow Corning, Wacker Chemie, and BASF, amongst others.

### Examples

Exemplary non-aqueous hair bleaching and coloring compositions in accordance with the present disclosure are set out in TABLE 2 below:

TABLE 2
Non-Aqueous Hair Bleaching and Coloring Compositions

| | | % by weight, actives | | |
|---|---|---|---|---|
| Ingredient¹ | Example source(s) | 1 | 2 | 3 |
| Sodium disilicate | GD ® Sodium Silicate | 22.0 | 15.0 | 21.0 |
| Sodium metasilicate | Simet ® FE | — | 5.0 | — |
| Fumed hydrophilic silica | Cabosil ® M 5 | 3.0 | 3.0 | 3.0 |
| Potassium persulfate | Misc. | 21.0 | 22.0 | 22.0 |
| Sodium persulfate | Misc. | — | — | 12.0 |
| Ammonium persulfate | Misc. | 20.0 | 20.0 | 6.0 |
| Mineral oil | Misc. | 24.0 | 24.5 | 25.0 |
| Acid dye(s) | Misc. | 0.5² | 1.5³ | 1.5⁴ |
| Polymer thickener | Carbopol ® 937 | 2.0 | 2.0 | 2.0 |
| Microbially-derived polysaccharide | Misc. | 1.5⁵ | 1.5⁵ | 1.5⁵ |
| C₈-C₂₆ carboxylate salt | Misc. | 3.5⁶ | 3.5⁶ | 3.5⁶ |
| EDTA-tetrasodium salt | Misc | 1.5 | 1.5 | 1.5 |
| Miscellaneous adjuvant | — | 1.0 | 0.5 | 1.0 |
| Total | | 100.0% | 100.0% | 100.0% |

| | | | |
|---|---|---|---|
| Specifications as measured | | | |
| Appearance (visual) | Red | Yellow | Purple |
| | Paste | Paste | Paste |
| Ammonia (as % NH₃ - distillation) | 3.0 | 3.0 | 0.9 |
| Active Oxygen (as % O₂—KMnO₄ titration) | 2.65 | 2.65 | 2.55 |
| Active Alkalinity (as % Na₂O to pH ~4.5) | 5.95 | 6.60 | 5.70 |
| Density (pycnometer @ 25° C.) | >1.25 | >1.25 | >1.25 |
| Viscosity (Brookfield, T-F, 25° C., cps × 10⁶) | 4.5 | 4.0 | 5.75 |

Superscripted notes in TABLE 2 are as follows: (1) All powdered ingredients used in the TABLE 2 compositions comprised particles having an average particle size of less than about 0.18 mm, or comprised only particles that passed through an 80 mesh Tyler Screen Mesh; (2) Acid Red 92 only; (3) Acid Yellow 1 only; (4) mixture of 0.5% Acid Red 92 and 1.0% Acid Violet 43; (5) xanthan gum; and (6) magnesium stearate.

Active oxygen was determined by standard potassium permanganate (KMnO₄) titration, and is reported in TABLE 2 as % O₂. Percent (%) ammonia was determined by distillation.

Compositions 1, 2, and 3 in TABLE 2 were titrated to an endpoint of pH ˜4.5, using an autotitrator, whereby it was determined that each composition in TABLE 2 had alkalinity of from about 5.0% to about 10.0% Na₂O-titratable alkalinity to pH ˜4.5. In various embodiments, compositions of the present disclosure may have alkalinity of less than about 10.0% Na₂O-titratable alkalinity to pH ˜4.5.

Viscosity was measured at 25° C. using a T-F T-Bar spindle, @ 0.1 rpm, on a Helipath Stand™ equipped Brookfield Viscometer, and is reported in TABLE 2 as “cps×10⁶”, that is, in millions of centipoise. As noted in TABLE 2, the compositions 1, 2, and 3 are very viscous, thick pastes, with Brookfield Helipath spindle viscosities from about 4,000,000 cps to about 6,500,000 cps. In various embodiments, compositions of the present disclosure may have viscosities of at least about 1,500,000 cps. In various embodiments, compositions of the present disclosure may have viscosities of from about 1,500,000 cps to about 6,500,000 cps. In various embodiments, compositions of the present disclosure may have viscosities of from about 4,000,000 cps to about 6,500,000 cps.

Compositions 1, 2, and 3 are opaque, rather than transparent, and are therefore “pastes” and not clear gels. In that sense, the products resemble colored toothpaste.

In various embodiments, a non-aqueous hair bleaching and coloring composition comprises: a silicate; a silica; a peroxy salt; a hydrocarbon; and a dye, wherein the % alkalinity of said composition is less than about 10% Na₂O titratable to pH ˜4.5, and wherein the viscosity of said composition is at least about 1,500,000 cps at 25° C. In various embodiments, said dye is selected from the group consisting of acid dyes, nonionic dyes, cationic dyes, and mixtures thereof. In various embodiments, said dye is an acid dye.

In various embodiments, a paste-like, non-aqueous hair bleaching and coloring composition comprises: a silicate; a silica; a peroxy salt; a microbially-derived polysaccharide; a polymer thickener; a hydrocarbon; and a dye, wherein each dry ingredient in said composition has an average particle size of less than about 0.18 mm or comprises only particles that pass through an 80 mesh Tyler Screen Mesh, wherein the % alkalinity of said composition is less than about 10% Na₂O titratable to pH ˜4.5, and wherein the viscosity of said composition is at least about 1,500,000 cps at 25° C.

In various embodiments, a paste-like, non-aqueous hair bleaching and coloring composition consists essentially of: a silicate; a silica; a peroxy salt; xanthan gum; a polyacrylate thickener; a hydrocarbon; and a dye, wherein each of said silicate, silica, peroxy salt, xanthan gum, polyacrylate thickener, and dye are present as dry powders, each having an average particle size of less than about 0.18 mm or comprising only particles that pass through an 80 mesh Tyler Screen Mesh, wherein the % alkalinity of said composition is less than about 10% Na₂O titratable to pH ˜4.5, and wherein the viscosity of said composition is at least about 1,500,000 cps at 25° C.

In various embodiments, a paste-like, non-aqueous hair bleaching and coloring composition consists essentially of: a silicate; a silica; a peroxy salt; xanthan gum; a polyacrylate thickener; a hydrocarbon; and a dye selected from the group consisting of acid dyes, nonionic dyes, cationic dyes, and mixtures thereof, wherein each of said silicate, silica, peroxy salt, xanthan gum, polyacrylate thickener, and dye are present as dry powders, each having an average particle size of less than about 0.18 mm or comprising only particles that pass through an 80 mesh Tyler Screen Mesh, wherein the % alkalinity of said composition is less than about 10% Na₂O titratable to pH ˜4.5, and wherein the viscosity of said composition is at least about 1,500,000 cps at 25° C.

In various embodiments, a paste-like, non-aqueous hair bleaching and coloring composition consists essentially of: a silicate; a silica; a peroxy salt; xanthan gum; a polyacrylate thickener; a hydrocarbon; and an acid dye, wherein each of said silicate, silica, peroxy salt, xanthan gum, polyacrylate thickener, and dye are present as dry powders, each having an average particle size of less than about 0.18 mm or comprising only particles that pass through an 80 mesh Tyler Screen Mesh, wherein the % alkalinity of said composition is less than about 10% Na₂O titratable to pH ˜4.5, and wherein the viscosity of said composition is at least about 1,500,000 cps at 25° C. In various embodiments, said composition has from about 5% to about 10% Na₂O titratable to pH ˜4.5. In various embodiments, said composition has a viscosity of from about 1,500,000 cps to about 6,500,000 at 25° C.

In various embodiments, a paste-like, non-aqueous hair bleaching and coloring composition consists essentially of: a silicate; a silica; a peroxy salt; xanthan gum; a polyacrylate thickener; a hydrocarbon; and an acid dye selected from the group consisting of Acid Yellow 1, Acid Yellow 24, Acid Red 92, Acid Violet 43, and mixtures thereof, wherein each of said silicate, silica, peroxy salt, xanthan gum, polyacrylate thickener, and acid dye are present as dry powders, each having an average particle size of less than about 0.18 mm or comprising only particles that pass through an 80 mesh Tyler Screen Mesh, wherein the % alkalinity of said composition is less than about 10% Na₂O titratable to pH ˜4.5, and wherein the viscosity of said composition is at least about 1,500,000 cps at 25° C. In various embodiments, said composition has from about 5% to about 10% Na₂O titratable to pH ˜4.5. In various embodiments, said composition has a viscosity of from about 1,500,000 cps to about 6,500,000 at 25° C.

Methods of Manufacturing

In various embodiments, such as for example, the Compositions 1-3 in TABLE 2, non-aqueous hair bleaching and coloring compositions in accordance to the present disclosure are generally prepared in accordance with a three-step process, comprising: (1) preparing a powder premix under defined conditions; (2) preparing a liquid premix under defined conditions; and (3) combining the powder premix from (1) with the liquid premix from (2) under defined conditions. In various embodiments, at least one dye is incorporated into the powder premix. In various embodiments, at least one dye is incorporated into the liquid premix. In various embodiments, dye is incorporated into the powder and/or liquid premix. In various embodiments, dye may be added after the liquid and powder premix are combined.

In various embodiments of the present disclosure, a method of manufacturing a non-aqueous hair bleaching and coloring composition comprises: producing a powder premix comprising a mixture of silicate, silica and peroxy salt; producing a liquid premix comprising a mixture of hydrocarbon and dye; and producing the non-aqueous hair bleaching and coloring composition by combining the powder premix and the liquid premix.

In various embodiments, the dye is chosen from the group consisting of anionic dyes, nonionic dyes, cationic dyes, and mixtures thereof.

In various embodiments, the powder premix further comprises additional dry and/or liquid ingredients chosen from the group consisting of surfactant, chelant, polymer thickener, microbially-derived polysaccharide, adjuvant, and mixtures thereof. In various embodiments, each of any additional dry ingredients incorporated into the powder premix has an average particle size of less than about 0.18 mm or comprises only particles that pass through an 80 mesh Tyler Screen Mesh.

In various embodiments, the liquid premix further comprises additional dry and/or liquid ingredients chosen from the group consisting of surfactant, chelant, polymer thickener, microbially-derived polysaccharide, adjuvant, and mixtures thereof. In various embodiments, each of any dry ingredients incorporated into the liquid premix has an average particle size of less than about 0.18 mm or comprises only particles that pass through an 80 mesh Tyler Screen Mesh.

In various embodiments of the method of manufacturing in accordance with the present disclosure, the powder premix comprises powdered ingredients each having an average particle size of less than about 0.18 mm, and/or powdered ingredients comprising only particles that pass through an 80 mesh Tyler Screen Mesh. In various embodiments of the method, the powdered ingredients having an average particle size of less than about 0.18 mm or comprising only particles that pass through an 80 mesh Tyler Screen Mesh are commercially sourced with this particle size distribution. In various embodiments of the method, the powdered ingredients are milled or ground in a suitable mill or grinder after purchase, such that each ingredient has an average particle size of less than about 0.18 mm or only particles that pass through an 80 mesh Tyler Screen Mesh prior to incorporation in the powder premix.

In various embodiments of the method, the actual blending of the powder premix, such as in a ribbon blender, can also reduce the particle size of any one or more of the dry ingredients to an average particle size of less than about 0.18 mm or only particles that pass through an 80 mesh Tyler Screen Mesh. Depending on the blending equipment and the speed and duration of the dry blending, particles in the blender may be broken down into smaller particles. In various embodiments of the method, the powder premix after blending is subsequently sieved such that the powder premix has average particle size of less than about 0.18 mm or only particles that pass through an 80 mesh Tyler Screen Mesh. In various embodiments of the method, the powder premix is blended in any suitable dry ingredient blender, such as for example, a ribbon blender, a double-cone blender, a Patterson-Kelley (“P-K”) blender, or the like. “Dry blending,” as the process is delineated herein, is not limited to the blending of only dry ingredients, as it is well known in chemical manufacturing that liquid ingredients may be poured or sprayed onto dry ingredients during the dry blending operation. For example, ribbon blender may have spray bars up inside them for application of a fine spray of liquid ingredients onto powders. In various embodiments, the dry blending is heated and/or cooled. In various embodiments, the ingredients for the powder premix may be added in any order, individually or in various combinations as appropriate, with intermittent mixing staged at any time, such as between or during additions of particular ingredients, and/or only when everything is combined in the blender.

In various embodiments of the method of manufacturing in accordance to the present disclosure, the liquid premix comprises hydrocarbon and at least one dye. In various embodiments, the hydrocarbon comprises any combination of liquids and waxes. In various embodiments, the dye is chosen from the group consisting of anionic dyes, nonionic dyes, cationic dyes, and mixtures thereof. In various embodiments, the dye is chosen from the group consisting of anionic dyes, nonionic dyes, and mixtures thereof. In various embodiments, the at least one dye comprises an acid dye. In various embodiments, the liquid premix includes only one or two acid dyes.

The liquid premix may be blended in any suitable kettle of any size, equipped for various processes such as heating, cooling, weighing, mixing, draining, and the like. For example, a commercial stainless steel mixing tank equipped with a heating jacket and an immersion mixer (e.g. a mixing blade/propeller on a long shaft) can suffice. In various embodiments, a sweep mixing assembly may be used. Sweep mixing utilizes a swing-motion rather than a rotating motion of the mixing arm/blade. In various embodiments, the mixing kettle is capable heating contents therein to temperatures from ambient up to at least about 200° C. During the production of the liquid premix, ingredients may be added in any order, with heating and/or mixing to any degree between or during various additions of ingredients, and/or after everything is in the kettle.

In various embodiments of the method of manufacturing, the liquid premix is prepared by adding the hydrocarbon comprising any combination of liquid hydrocarbon and waxes, along with other ingredients such as surfactant to the kettle and heating and mixing at from about 95° to about 125° C. In various embodiments, the heating is sufficient to melt any waxy hydrocarbons that may be incorporated into the liquid premix. The agitation of these ingredients may be continuous and at moderate, low sheer speed. This mixing may be carried on until the mixture in the kettle is clear and homogeneous. In various embodiments, this hydrocarbon mixture is then allowed to cool and/or cooled by external means to an internal temperature of from about 50° to about 60° C. at which time the dye is added. In various embodiments of the method, the dye has an average particle size of less than about 0.18 mm or comprises only particles that pass through an 80 mesh Tyler Screen Mesh. Additional ingredients, dry and/or liquid in nature, may be added with and/or after the dye. In various embodiments of the method, the hydrocarbon/dye mixture may be further mixed at any time/temperature until a homogeneous liquid premix is obtained. The hydrocarbon/dye mixture is also referred to herein as the “dyed liquid premix” to distinguish from a liquid premix comprising hydrocarbon and optionally other ingredients except dyes.

In various embodiments, the method of manufacturing a non-aqueous hair bleaching and coloring composition comprises combining the powder premix with the liquid premix. In various embodiments, the liquid premix may be added to the powder premix, or vice versa. In various embodiments, the liquid premix is transferred to a mixing drum where it may cool and congeal. In various embodiments, the powder premix may be added to the congealed liquid premix in portions while mixing with a dual auger planetary mixer, spiral mixer, or any other suitable blending apparatus, to obtain the non-aqueous hair bleaching and coloring composition in the form of a paste-like mixture. In this way, very thick and colored pastes having viscosity greater than about 1,500,000 cps (measured at 25° C.) are obtainable.

In various embodiments, a method of manufacturing a non-aqueous hair bleaching and coloring composition comprises: (1) producing a powder premix by blending a silicate, a silica, and a peroxy salt at ambient temperature, wherein each of said silicate, silica and peroxy salt has an average particle size of less than about 0.18 mm or comprises only particles that pass through an 80 mesh Tyler Screen Mesh; (2) producing a liquid premix by mixing and heating at from about 95° to about 125° C. a hydrocarbon and a dye; (3) producing a congealed liquid premix by cooling said liquid premix from about ambient temperature to about 65° C. until said liquid premix congeals; and (4) combining said powder premix with said congealed liquid premix at from about ambient temperature to about 50° C. while mixing to obtain said non-aqueous hair bleaching and coloring composition in the form of a paste.

In various embodiments, the step of producing a powder premix further comprises the addition of other ingredients selected from the group consisting of surfactant, chelant, polymer thickener, microbially-derived polysaccharide, adjuvant, and mixtures thereof. In various embodiments, any ingredients from this group that are added in powdered form have an average particle size of less than about 0.18 mm or comprise only particles that pass through an 80 mesh Tyler Screen Mesh. In various embodiments, the step of producing a liquid premix further comprises the addition of other ingredients selected from the group consisting of surfactant, chelant, polymer thickener, microbially-derived polysaccharide, adjuvant, and mixtures thereof. In various embodiments, any ingredients from this group that are added in powdered form have an average particle size of less than about 0.18 mm or comprise only particles that pass through an 80 mesh Tyler Screen Mesh.

In various embodiments, a method of manufacturing a non-aqueous hair bleaching and coloring composition comprises: (1) producing a powder premix by blending a silicate, a silica, a peroxy salt, optional microbially-derived polysaccharide, optional polymer thickener, and optional adjuvant, at ambient temperature, wherein each dry ingredient has an average particle size of less than about 0.18 mm or comprises only particles that pass through an 80 mesh Tyler Screen Mesh; (2) producing a liquid premix by mixing, and heating at from about 95° to about 125° C., hydrocarbon chosen from the group consisting of liquid hydrocarbon, waxes, and mixtures thereof, with optional surfactant; (3) producing a dyed liquid premix by cooling said liquid premix to about 50° to about 65° C. and adding at least one dye; (4) congealing said dyed liquid premix by cooling said dyed liquid premix to about ambient temperature to about 50° C. until said dyed liquid premix congeals; and (5) combining said powder premix with said congealed dyed liquid premix at from about ambient temperature to about 50° C., while mixing, to obtain said non-aqueous hair bleaching and coloring composition in the form of a paste.

For producing the examples in TABLE 2, a method of manufacturing non-aqueous hair bleaching and coloring compositions comprises:

(1) producing a powder premix by blending silicate, silica, peroxy salt, chelant, microbially-derived polysaccharide, polymer thickener, and optional adjuvant at ambient temperature until uniform, wherein each ingredient in dry form has an average particle size of less than about 0.18 mm or comprises only particles that pass through an 80 mesh Tyler Screen Mesh;

(2) producing a liquid premix by mixing and heating at from about 95° to about 125° C. a hydrocarbon and a C₈-C₂₆ carboxylate salt, said hydrocarbon chosen from the group consisting of liquid hydrocarbon, waxes, and mixtures thereof;

(3) cooling said liquid premix to about 50° to about 65° C. while continuously mixing;

(4) producing a dyed liquid premix by adding at least one dye to said liquid premix and mixing further for at least about 1 hour;

(5) congealing said dyed liquid premix by cooling said dyed liquid premix to about 40° C.; and

(6) adding said powder premix into said congealed dyed liquid premix and mixing with a dual auger planetary mixer continuously for between about 10 minutes and about 25 minutes,

wherein said method produces said non-aqueous hair bleaching and coloring composition in the form of a uniform paste having total alkalinity at from about 5.0% to about 10.0% Na₂O-titratable to pH=4.5 and a viscosity of at least 1,500,000 cps at 25° C.

In various embodiments, the hydrocarbon in the method immediately above may consist essentially of mineral oil. In various embodiments, the at least one dye in the method immediately above may comprise any dye selected from the group consisting of anionic dyes, nonionic dyes, cationic dyes, and mixtures thereof. In various embodiments, the at least one dye in the method immediately above may comprise at least one acid dye. In various embodiments, the dye in the method immediately above is selected from the group consisting of Acid Yellow 1, Acid Yellow 24, Acid Red 92, Acid Violet 43, Disperse Red 17, tetrabromophenol blue, and mixtures thereof. In various embodiments, the dye in the method immediately above is selected from the group consisting of Acid Yellow 1, Acid Yellow 24, Acid Red 92, Acid Violet 43, tetrabromophenol blue, and mixtures thereof. In various embodiments, the dye in the method immediately above is selected from the group consisting of Acid Yellow 1, Acid Red 92, Acid Violet 43, and mixtures thereof.

Methods of Use

In various embodiments, compositions in accordance to the present disclosure may be mixed with aqueous peroxide to form an activated composition prior to application to the hair. In various embodiments, compositions in accordance to the present disclosure may be mixed with aqueous hydrogen peroxide to form an activated composition prior to application to the hair. The paste-like consistency of the compositions of the present disclosure, such as produced by methods of manufacturing in accordance to the present disclosure, blend easily and efficiently with aqueous peroxide solutions without clumping or separation occurring prior to, and/or during, application to the hair. The activated mixture comprising a composition of the present disclosure and aqueous peroxide uniformly bleaches and colors the hair shafts, with the uniformity of these processes due at least in part to the uniform nature of the non-aqueous hair bleaching and coloring compositions comprising carefully screened particle sizes for the dry ingredients, as discussed above.

In various embodiments, a method of simultaneously bleaching and coloring hair comprises forming an activated mixture from any of the non-aqueous bleaching and coloring compositions herein with aqueous peroxide and applying the activated mixture to the hair.

It will be apparent to those skilled in the art that various modifications and variations can be made in the present disclosure without departing from the spirit or scope of the disclosure. Thus, it is intended that the present disclosure cover the modifications and variations of this disclosure provided they come within the scope of the appended claims and their equivalents.

Likewise, numerous characteristics and advantages have been set forth in the preceding description, including various alternatives together with details of the structure and function of the devices and/or methods. The disclosure is intended as illustrative only and as such is not intended to be exhaustive. It will be evident to those skilled in the art that various modifications may be made, especially in matters of structure, materials, elements, components, shape, size and arrangement of parts including combinations within the principles of the disclosure, to the full extent indicated by the broad, general meaning of the terms in which the appended claims are expressed. To the extent that these various modifications do not depart from the spirit and scope of the appended claims, they are intended to be encompassed therein.

What is claimed is:

1. A non-aqueous hair bleaching and coloring composition comprising:
a. a silicate;
b. a silica;
c. a peroxy salt;
d. a hydrocarbon; and
e. a dye,
wherein the % alkalinity of said composition is less than about 10% Na₂O titratable to pH ˜4.5, and wherein the viscosity of said composition is at least about 1,500,000 cps at 25° C.

2. The composition according to claim 1, wherein said hydrocarbon comprises a C₁₀-C₁₀₀ linear, branched, saturated, partly unsaturated, cyclic, and/or aromatic hydrocarbon.

3. The composition according to claim 1, wherein said hydrocarbon comprises mineral oil.

4. The composition according to claim 1, wherein said hydrocarbon is present at from about 15.0% by weight to about 40.0% by weight, based on the total weight of the composition.

5. The composition of claim 1, wherein said composition is devoid of any organic esters.

6. The composition of claim 1 wherein the peroxy salt is selected from the group consisting of sodium, potassium, and/or ammonium persulfate, percarbonate, perborate, peroxide, and mixtures thereof.

7. The composition of claim 1, wherein said peroxy salt is present at from about 25.0 by weight to about 60.0% by weight, based on the total weight of the composition.

8. The composition of claim 1 wherein said silicate is a sodium and/or potassium silicate having SiO₂/M₂O ratio of from about 0.75 to about 2.25.

9. The composition of claim 1, where said alkalinity is from about 5.0% to about 10.0%.

10. The composition of claim 1, wherein each of said silicate, silica, peroxy salt and dye comprise particles of average size less than about 0.180 mm, or comprise particles that pass through a Tyler 80 mesh screen.

11. The composition of claim 1, wherein said silica is present at from about 2.0% by weight to about 4.0% by weight, based on the total weight of the composition.

12. The composition of claim 1, wherein said silica is chosen from the group consisting of hydrophilic untreated silica, hydrophobic-treated silica, and mixtures thereof.

13. The composition of claim 1, wherein said dye is present at from about 0.01% by weight to about 7.00% by weight, based on the total weight of the composition.

14. The composition of claim 1, wherein said dye is selected from the group consisting of acid dyes, nonionic dyes, cationic dyes, and mixtures thereof.

15. The composition of claim 1, wherein said dye is selected from the group consisting of acid dyes, nonionic dyes, and mixtures thereof.

16. The composition of claim 1, wherein said dye is an acid dye.

17. The composition of claim 16, wherein said acid dye is selected from the group consisting of Acid Yellow 1, Acid Yellow 24, Acid Red 92, Acid Violet 43, tetrabromophenol blue, and mixtures thereof.

18. The composition of claim 16, wherein said acid dye is selected from the group consisting of Acid Yellow 1, Acid Yellow 24, Acid Yellow 73, Acid Red 92, Acid Violet 43, tetrabromophenol blue, and mixtures thereof

19. The composition of claim 1, wherein said dye is selected from the group consisting of Acid Yellow 1, Acid Yellow 24, Acid Red 92, Acid Violet 43, Disperse Red 17, tetrabromophenol blue, and mixtures thereof.

20. The composition of claim 1, wherein said dye is selected from the group consisting of Acid Yellow 1, Acid Yellow 24, Acid Yellow 73, Acid Red 92, Acid Violet 43, Disperse Red 17, tetrabromophenol blue, and mixtures thereof.

21. A paste-like, non-aqueous hair bleaching and coloring composition comprising:
a. a silicate;
b. a silica;
c. a peroxy salt;
d. a microbially-derived polysaccharide;
e. a polymer thickener;
f. a hydrocarbon; and
g. a dye,
wherein each dry ingredient in said composition has an average particle size of less than about 0.18 mm or comprises only particles that pass through an 80 mesh Tyler Screen Mesh,
wherein the % alkalinity of said composition is less than about 10% Na₂O titratable to pH ˜4.5, and
wherein the viscosity of said composition is at least about 1,500,000 cps at 25° C.

22. The composition of claim 21, wherein said dye comprises an acid dye.

23. The composition of claim 21, wherein said dye is selected from the group consisting of Acid Yellow 1, Acid Yellow 24, Acid Red 92, Acid Violet 43, Disperse Red 17, tetrabromophenol blue, and mixtures thereof.

24. The composition of claim 21, wherein said dye is selected from the group consisting of Acid Yellow 1, Acid Yellow 24, Acid Yellow 73, Acid Red 92, Acid Violet 43, Disperse Red 17, tetrabromophenol blue, and mixtures thereof.

25. The composition of claim 21, wherein said hydrocarbon consists essentially of mineral oil.

26. The composition of claim 21, wherein said viscosity is from about 1,500,000 cps to about 6,500,000 cps at 25° C.

27. The composition of claim 21, wherein said alkalinity is from about 5.0% to about 10% Na₂O titratable to pH ˜4.5.

28. A method of manufacturing a non-aqueous hair bleaching and coloring composition, said method comprising:
a. producing a powder premix by blending a silicate, a silica, and a peroxy salt at ambient temperature, wherein each of said silicate, silica and peroxy salt has an average particle size of less than about 0.18 mm or comprises only particles that pass through an 80 mesh Tyler Screen Mesh;
b. producing a liquid premix by mixing and heating to a temperature at from about 95° to about 125° C. a hydrocarbon and a dye;
c. producing a congealed liquid premix by cooling said liquid premix to a temperature at from about ambient temperature to about 65° C. until said liquid premix congeals; and
d. combining said powder premix with said congealed liquid premix at from about ambient temperature to about 50° C. while mixing to obtain said non-aqueous hair bleaching and coloring composition in the form of a paste.

29. The method of claim 28, wherein the step of producing the powder premix further comprises the addition of other ingredients selected from the group consisting of surfactant, chelant, polymer thickener, microbially-derived polysaccharide, adjuvant, and mixtures thereof.

30. The method of claim 29, wherein each of said other ingredients incorporated as dry powders have an average particle size of less than about 0.18 mm or comprise only particles that pass through an 80 mesh Tyler Screen Mesh.

31. The method of claim 28, wherein the step of producing the liquid premix further comprises the addition of other ingredients selected from the group consisting of surfactant, chelant, polymer thickener, microbially-derived polysaccharide, adjuvant, and mixtures thereof.

32. The method of claim 31, wherein each of said other ingredients incorporated as dry powders have an average particle size of less than about 0.18 mm or comprise only particles that pass through an 80 mesh Tyler Screen Mesh.

33. The method of claim 28, wherein said dye is selected from the group consisting of acid dyes, nonionic dyes, cationic dyes, and mixtures thereof.

34. The method of claim 28, wherein said dye is an acid dye.

35. The method of claim 28, wherein said dye is selected from the group consisting of Acid Yellow 1, Acid Yellow 24, Acid Red 92, Acid Violet 43, Disperse Red 17, tetrabromophenol blue, and mixtures thereof.

36. The method of claim 28, wherein said dye is selected from the group consisting of Acid Yellow 1, Acid Yellow 24, Acid Yellow 73, Acid Red 92, Acid Violet 43, Disperse Red 17, tetrabromophenol blue, and mixtures thereof.

37. A method of manufacturing a non-aqueous hair bleaching and coloring composition, said method comprising:
a. producing a powder premix by blending a silicate, a silica, a peroxy salt, optional microbially-derived polysaccharide, optional polymer thickener, and optional adjuvant, at ambient temperature, wherein each dry ingredient has an average particle size of less than about 0.18 mm or comprises only particles that pass through an 80 mesh Tyler Screen Mesh;
b. producing a liquid premix by mixing, and heating at from about 95° to about 125° C., hydrocarbon chosen from the group consisting of liquid hydrocarbon, waxes, and mixtures thereof, with optional surfactant;
c. producing a dyed liquid premix by cooling said liquid premix to about 50° to about 65° C. and adding at least one dye;
d. congealing said dyed liquid premix by cooling said dyed liquid premix to about ambient temperature to about 50° C. until said dyed liquid premix congeals; and
e. combining said powder premix with said congealed dyed liquid premix at from about ambient temperature to about 50° C., while mixing, to obtain said non-aqueous hair bleaching and coloring composition in the form of a paste,
wherein said non-aqueous hair bleaching and coloring composition has a viscosity of at least about 1,500,000 cps at 25° C.

38. A method of manufacturing a non-aqueous hair bleaching and coloring composition, said method comprising:
a. producing a powder premix by blending silicate, silica, peroxy salt, chelant, microbially-derived polysaccharide, polymer thickener, and optional adjuvant at ambient temperature until uniform, wherein each ingredient in dry form has an average particle size of less than about 0.18 mm or comprises only particles that pass through an 80 mesh Tyler Screen Mesh;
b. producing a liquid premix by mixing and heating at from about 95° to about 125° C. a hydrocarbon and a C₈-C₂₆ carboxylate salt, said hydrocarbon chosen from the group consisting of liquid hydrocarbon, waxes, and mixtures thereof;
c. cooling said liquid premix to about 50° to about 65° C. while continuously mixing;
d. producing a dyed liquid premix by adding at least one dye to said liquid premix and mixing further for at least about 1 hour;
e. congealing said dyed liquid premix by cooling said dyed liquid premix to about 40° C.; and
f. adding said powder premix into said congealed dyed liquid premix and mixing with a dual auger planetary mixer continuously for between about 10 minutes and about 25 minutes,
wherein said method produces said non-aqueous hair bleaching and coloring composition in the form of a uniform, thick paste having a total alkalinity at from about 5.0% to about 10.0% Na₂O-titratable to pH ˜4.5 and a viscosity of at least about 1,500,000 cps at 25° C.

39. A method of simultaneously bleaching and coloring hair, said method comprising: forming an activated mixture by combining the composition of claim 1 with aqueous peroxide; and applying said activated mixture to hair.

40. A method of simultaneously bleaching and coloring hair, said method comprising: forming an activated mixture by combining the composition of claim 21 with aqueous peroxide; and applying said activated mixture to hair.
